(19) United States
(12) Patent Application Publication
(10) Pub. No.: US 2021/0257059 A1
(43) Pub. Date: Aug. 19, 2021

(54) INFORMATION PROCESSING APPARATUS, INFORMATION PROCESSING METHOD, AND PROGRAM

(71) Applicant: SONY CORPORATION, TOKYO (JP)

(72) Inventor: KAORU YOSHIDA, TOKYO (JP)

(21) Appl. No.: 17/250,206

(22) PCT Filed: Jun. 11, 2019
(86) PCT No.: PCT/JP2019/023117

§ 371 Date: Dec. 14, 2020

(30) Foreign Application Priority Data
Jun. 22, 2018 (JP) 2018-119079

(51) Int. Cl. G16B 45/00; G16B 40/20; G16B 10/00

(52) U.S. Cl. CPC G16B 45/00; G16B 10/00; G16B 40/20

(57) ABSTRACT

To analyze relationship between families of homologous domains in different classification categories and relationship between classification categories based on the relationship between the families using an analysis result obtained through a homologous domain phylogeny analysis method. An information processing apparatus (10) according to the present disclosure includes: a homologous domain family bunch classifying unit (103) configured to classify a group of families having homology across classification categories as a homologous domain family bunch for a plurality of pieces of homologous domain family information belonging to a plurality of the classification categories on the basis of homologous domain family information regarding the families of homologous string pieces and content of the homologous string pieces belonging to the families included in a result of phylogeny analysis by utilizing the result of phylogeny analysis performed on the basis of positions where the homologous string pieces exist and homological relationship of the homologous string pieces while attention is focused on the homologous string pieces within a string for string data representing the string including one or more characters.

### FIELD

The present disclosure relates to an information processing apparatus, an information processing method, and a program.

### BACKGROUND

While inheriting between a parent and a child of living things (cells) is called vertical transfer, exchange between living things (cells) of the same type or different types is called horizontal transfer. The horizontal transfer is posteriori acquisition of genetic information and is considered to contribute to diversity such as continuation of species, including adaptation to an environment, toxicity, self-defense, and the like. Here, a biological mechanism in which genetic information transfers between cells (genomes) or within a cell includes ingression in which fragmented genetic information is absorbed, infection via phages, viruses, or the like, having packages, coupling which is transfer by cell binding, and the like.

In contrast with vertical transfer, change of genetic information (particularly, copy, transfer and mutation of genetic information) within a cell (genome) also in horizontal transfer has started to be known by study of transfer elements of corns by Barbara McClintock. Further, also in drosophilae, transfer elements called transposons have been actively studied, and deep involvement between transfer elements and gene expression control has become obvious.

In molecular evolutionary biology, a phylogenetic tree which indicates a time point at which each living thing has emerged has been shown so far on the basis of slight mutation within genes such as 16S rRNA which are common genes possessed by all living things. However, such a method cannot explain evolutionary relationship of living things well because the method does not reflect horizontal transfer as described above at all, and, in addition, mutation speed, and eventually, a phylogenetic tree becomes different depending on a gene on which attention is focused.

The present inventor therefore proposes a method (homologous domain phylogeny analysis method) of extracting all homologous domains within genomes from a whole genome sequence including chromosomes and plasmids of one living thing only through self-alignment instead of alignment with other genome sequences, and autonomously structuring and constructing phylogeny on the basis of homology and geographical proximity of the extracted all homologous domains within the genomes (see Patent Literature 1 below). This homologous domain phylogeny analysis method is different from the phylogeny analysis method in related art which is based on slight mutation within genes as described above, is a phylogeny analysis method which focuses attention on genetic information which is less shared by living things and which appears a plurality of times, and excels in extraction of genetic information obtained through horizontal transfer. Further, this homologous domain phylogeny analysis method is a method having a wide variety of uses which can be utilized in structural analysis of sequential data including all characters, signs and numerical values, like music, video, economic trend, and the like, as well as genome sequences.

### CITATION LIST

### Patent Literature

Patent Literature 1: JP 2012-9008 A

### SUMMARY

### Technical Problem

Here, the homologous domain phylogeny analysis method proposed in the above-described Patent Literature 1 is a method of structuring and constructing phylogeny of the extracted homologous domains while attention is focused on homologous domains within the same strain regarding a “strain” which is one of biological classification categories. Therefore, the method cannot sufficiently discuss relationship between homologous domains in different strains (for example, relationship between a family of homologous domains in a certain strain A and a family of homologous domains in a strain B which is different from the strain A). Study of relationship between families of homologous domains across classification categories is important in construction of a phylogenetic tree of biological evolution.

Therefore, in view of the above-described circumstances, the present inventor proposes an information processing apparatus, an information processing method, and a program which are capable of analyzing relationship between families of homologous domains in different classification categories and relationship between classification categories based on the relationship between the families using an analysis result obtained through a homologous domain phylogeny analysis method.

### Solution to Problem

According to the present disclosure, an information processing apparatus is provided that includes: a homologous domain family bunch classifying unit configured to classify a group of families having homology across classification categories as a homologous domain family bunch for a plurality of pieces of homologous domain family information belonging to a plurality of the classification categories on the basis of homologous domain family information regarding the families of homologous string pieces and content of the homologous string pieces belonging to the families included in a result of phylogeny analysis by utilizing the result of phylogeny analysis performed on the basis of positions where the homologous string pieces exist and homological relationship of the homologous string pieces while attention is focused on the homologous string pieces within a string for string data representing the string including one or more characters.

Moreover, according to the present disclosure, an information processing method is provided that includes: classifying a group of families having homology across classification categories as a homologous domain family bunch for a plurality of pieces of homologous domain family information belonging to a plurality of the classification categories on the basis of homologous domain family information regarding the families of homologous string pieces and content of the homologous string pieces belonging to the families included in a result of phylogeny analysis by utilizing the result of phylogeny analysis performed on the basis of positions where the homologous string pieces exist and homological relationship of the homologous string pieces while attention is focused on the homologous string pieces within a string for string data representing the string including one or more characters.

Moreover, according to the present disclosure, a program is provided that causes a computer to realize: a homologous domain family bunch classifying function of classifying a group of families having homology across classification categories as a homologous domain family bunch for a plurality of pieces of homologous domain family information belonging to a plurality of the classification categories on the basis of homologous domain family information regarding the families of homologous string pieces and content of the homologous string pieces belonging to the families included in a result of phylogeny analysis by utilizing the result of phylogeny analysis performed on the basis of positions where the homologous string pieces exist and homological relationship of the homologous string pieces while attention is focused on the homologous string pieces within a string for string data representing the string including one or more characters.

According to the present disclosure, the homologous domain family bunch classifying unit classifies a group of families having homology across classification categories as a homologous domain family bunch on the basis of the homologous domain family information for a plurality of pieces of homologous domain family information belonging to a plurality of classification categories.

### Advantageous Effects of Invention

According to the present disclosure as described above, it becomes possible to analyze relationship between families of homologous domains in different classification categories and relationship between classification categories based on the relationship between the families using an analysis result obtained through a homologous domain phylogeny analysis method.

Note that the effects described above are not necessarily limitative. With or in the place of the above effects, there may be achieved any one of the effects described in this specification or other effects that may be grasped from this specification.

### BRIEF DESCRIPTION OF DRAWINGS

FIG. 1 is a block diagram illustrating an example of a configuration of an information processing apparatus according to an embodiment of the present disclosure.

FIG. 2 is a block diagram illustrating an example of a configuration of a homologous domain family bunch classifying unit of the information processing apparatus according to the embodiment.

FIG. 3 is an explanatory diagram for explaining an example of homologous domain family information in the embodiment.

FIG. 4 is an explanatory diagram for explaining an example of homologous domain family information in the embodiment.

FIG. 5 is an explanatory diagram for explaining an example of homologous domain family information in the embodiment.

FIG. 6 is an explanatory diagram for explaining sequence alignment processing in the embodiment.

FIG. 7 is an explanatory diagram for explaining a homologous domain family bunch in the embodiment.

FIG. 8 is a flowchart illustrating an example of flow of an information processing method according to the embodiment.

FIG. 9 is a block diagram illustrating an example of a modified example of the information processing apparatus according to the embodiment.

FIG. 10 is a block diagram illustrating an example of a hardware configuration of the information processing apparatus according to the embodiment.

FIG. 11 is an explanatory diagram for explaining an example.

FIG. 12 is an explanatory diagram for explaining an example.

FIG. 13 is an explanatory diagram for explaining an example.

FIG. 14 is an explanatory diagram for explaining an example.

FIG. 15 is an explanatory diagram for explaining an example.

### DESCRIPTION OF EMBODIMENTS

Favorable embodiments of the present disclosure will be described in detail with reference to the appended drawings. In this specification and the drawings, components having substantially the same functional configuration are denoted by the same reference numerals, and redundant description is omitted.

The description will be given in the following order.

1. Embodiment

1.1. Overall configuration of information processing apparatus

1.2. Configuration of homologous domain family bunch classifying unit

1.3. Modified example of information processing apparatus

1.4. Flow of information processing method

1.5. Hardware configuration of information processing apparatus

2. Examples

### Embodiment

<Overall Configuration of Information Processing Apparatus>

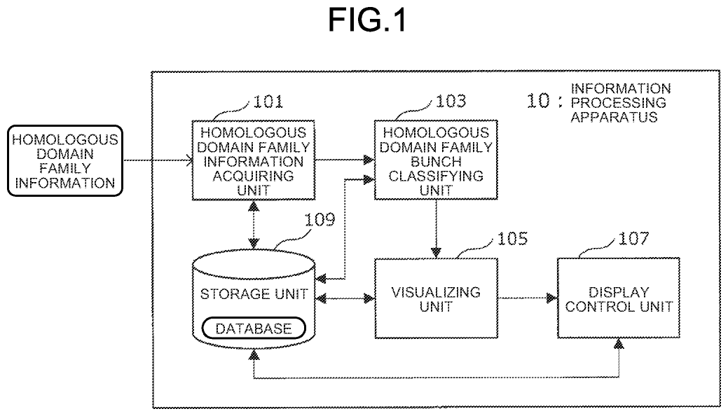

First, an overall configuration of an information processing apparatus according to an embodiment of the present disclosure will be described in detail with reference to FIG. 1. FIG. 1 is a block diagram illustrating an example of the configuration of the information processing apparatus according to the present embodiment.

An information processing apparatus 10 according to the present embodiment is an apparatus which analyzes relationship between families of homologous domains in different classification categories using an analysis result obtained through a homologous domain phylogeny analysis method. In the homologous domain phylogeny analysis method, a target string is aligned with itself, and a pair of sequences in a section (homologous section) linked by the relationship of homology (homological relationship) is extracted. Thereafter, in the homologous domain phylogeny analysis method, a bunch (homological group) in homologous section linked by one or more homological relationship chains and a region (regional group) in which homologous sections are regionally adjacent to or overlapped with each other are extracted, and a combined group of the homological group and the regional group is defined as a family. Hereinafter, the above-described homologous section and the homologous region will be collectively referred to as a homologous domain.

As schematically illustrated in FIG. 1, the information processing apparatus 10 according to the present embodiment mainly includes a homologous domain family information acquiring unit 101, a homologous domain family bunch classifying unit 103, a visualizing unit 105, a display control unit 107, and a storage unit 109.

The homologous domain family information acquiring unit 101 is realized with, for example, a central processing unit (CPU), a read only memory (ROM), a random access memory (RAM), a communication device, and the like. The homologous domain family information acquiring unit 101 acquires data of homologous domain family information which is to be used to analyze relationship between families of homologous domains in different classification categories at the information processing apparatus 10 according to the present embodiment. Here, the homologous domain family information is obtained by utilizing a result which is obtained through phylogeny analysis (homologous domain phylogeny analysis) performed on the basis of positions where homologous string pieces exist and homological relationship of the homologous string pieces while attention is focused on the homologous string pieces within a string for string data indicating the string including one or more characters. In this homologous domain family information, a homologous domain family of homologous string pieces (corresponding to a “large family” in the above-described Patent Literature 1, hereinafter, may be abbreviated as a “family”) and content of the homologous string pieces belonging to the family, included in the phylogeny analysis result are associated with each other.

The homologous domain family information acquiring unit 101 acquires the homologous domain family information as described above from a computer (not illustrated), or the like, which performs the homologous domain phylogeny analysis method as disclosed in, for example, Patent Literature 1, and outputs the acquired homologous domain family information to the homologous domain family bunch classifying unit 103 disposed downstream. Further, the homologous domain family information acquiring unit 101 may store the acquired homologous domain family information in the storage unit 109, or the like, disposed downstream, in association with time information regarding date, time, and the like, at which the homologous domain family information is acquired.

Here, the homologous domain family information as described above is obtained by analyzing, with the homologous domain phylogeny analysis method, sequential data including various kinds of characters, signs, numerical values, and the like, like genome sequences, music, video, economic trend, and the like. Hereinafter, description will be provided using an example of a case where relationship between homologous domain family information regarding genome sequences in different strains is analyzed while attention is focused on string data (that is, genome sequences) representing biological genetic information and a “strain” which is one of biological classification categories.

The homologous domain family bunch classifying unit 103 is realized with, for example, a CPU, a ROM, a RAM, an output device, a communication device, and the like. The homologous domain family bunch classifying unit 103 analyzes a plurality of pieces of homologous domain family information belonging to a plurality of classification categories, and classifies a group of families “having homology across classification categories (for example, strains of living things (particularly, cells))” for the plurality of pieces of homologous domain family information belonging to the plurality of classification categories, as a “homologous domain family bunch (global bunch)”. In this classification processing, the homologous domain family bunch classifying unit 103 can utilize various kinds of databases, software, or the like, stored in the storage unit 109, and the like, which will be described later, various kinds of databases, software, or the like, provided outside the information processing apparatus 10 or on a network as appropriate. Details of the classification processing performed at such a homologous domain family bunch classifying unit 103 will be described again later.

The homologous domain family bunch classifying unit 103 can output information regarding a homologous domain family bunch obtained through predetermined classification processing from the plurality of pieces of homologous domain family information belonging to the plurality of classification categories, to various kinds of externally provided servers, computers, or the like, as electronic data, can store the information in various kinds of information recording media as databases, or can output the information as paper, or the like. Further, the homologous domain family bunch classifying unit 103 can also visualize at least part of information of the obtained homologous domain family bunch or a database which is an aggregate of information regarding the obtained homologous domain family bunch, with the visualizing unit 105 which will be described later, and can display the information at a display apparatus such as a display provided at the information processing apparatus 10 or a display apparatus such as a display provided outside the information processing apparatus 10. Still further, the homologous domain family bunch classifying unit 103 preferably stores the information regarding the obtained homologous domain family bunch in the storage unit 109, or the like, disposed downstream as a database in association with time information regarding date, time, and the like, at which the information regarding the homologous domain family bunch is generated.

The visualizing unit 105 is realized with, for example, a CPU, a ROM, a RAM, an output device, a communication device, and the like. The visualizing unit 105 visualizes a plurality of homologous domain family bunches classified by the homologous domain family bunch classifying unit 103 on the basis of relationship of belonging of the homologous domain family bunches to the classification categories. Visualization of the homologous domain family bunches based on the relationship of belonging to the classification categories allows relationship among the plurality of homologous domain family bunches to be visually recognized, so that a user of the information processing apparatus 10 can easily grasp relationship among the plurality of homologous domain family bunches.

Further, the visualizing unit 105 can information-geometrically visualize analogous relationship among classification categories by performing at least one of principal component analysis (PCA) on the homologous domain family bunches belonging to the classification categories or multi-dimensional scaling (MDS), separately from visualization as described above. Use of such an analysis method enables relationship among the classification categories to be visualized more easily. Specific examples of visualization of relationship among the plurality of homologous domain family bunches by the visualizing unit 105 will be described again in examples which will be described below. Further, the visualizing unit 105 may utilize a known application such as Graphviz to visualize relationship among the plurality of homologous domain family bunches.

The visualizing unit 105 can output visualized information regarding relationship among the plurality of homologous domain family bunches to various kinds of externally provided servers, computers, or the like, as electronic data, can store the information in various kinds of information recording media, or can output the information as paper, or the like. Further, the visualizing unit 105 can also display the obtained visualized information at a display apparatus such as a display provided at the information processing apparatus 10 or at a display apparatus such as a display provided outside the information processing apparatus 10 via the display control unit 107. Still further, the visualizing unit 105 may store the obtained visualized information in the storage unit 109, or the like, disposed downstream, as a database in association with time information regarding date, time, and the like, at which the visualized information is generated.

The display control unit 107 is realized with, for example, a CPU, a ROM, a RAM, an output device, a communication device, and the like. The display control unit 107 performs display control to display information itself regarding the homologous domain family bunches generated by the homologous domain family bunch classifying unit 103 or the visualized information regarding the homologous domain family bunches generated by the visualizing unit 105 at an output device such as a display provided at the information processing apparatus 10, an output device provided outside the information processing apparatus 10, or the like. The user of the information processing apparatus 10 can thereby grasp various kinds of information regarding the homologous domain family bunches on the spot.

The storage unit 109 is realized with, for example, a RAM, a storage device, or the like, provided at the information processing apparatus 10 according to the present embodiment. The storage unit 109 stores various kinds of databases, software programs, and the like, which are to be utilized by the homologous domain family bunch classifying unit 103 to perform classification processing. Further, the storage unit 109 stores various kinds of setting information for processing of classifying homologous domain family bunches, processing of visualizing homologous domain family bunches, and the like, various parameters which are required to be stored when the information processing apparatus 10 according to the present embodiment performs some kinds of processing, a progress report of the processing, and the like, as appropriate. The homologous domain family information acquiring unit 101, the homologous domain family bunch classifying unit 103, the visualizing unit 105, the display control unit 107, and the like, can freely read/write data to/from this storage unit 109.

The overall configuration of the information processing apparatus 10 according to the present embodiment has been described in detail above with reference to FIG. 1.

<Configuration of Homologous Domain Family Bunch Classifying Unit>

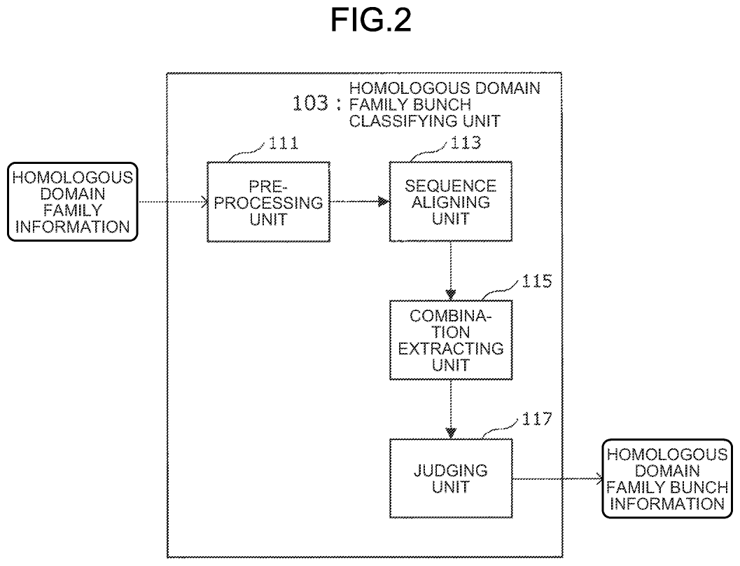

A configuration of the homologous domain family bunch classifying unit 103 provided at the information processing apparatus 10 according to the present embodiment will be described in detail next with reference to FIG. 2 to FIG. 7.

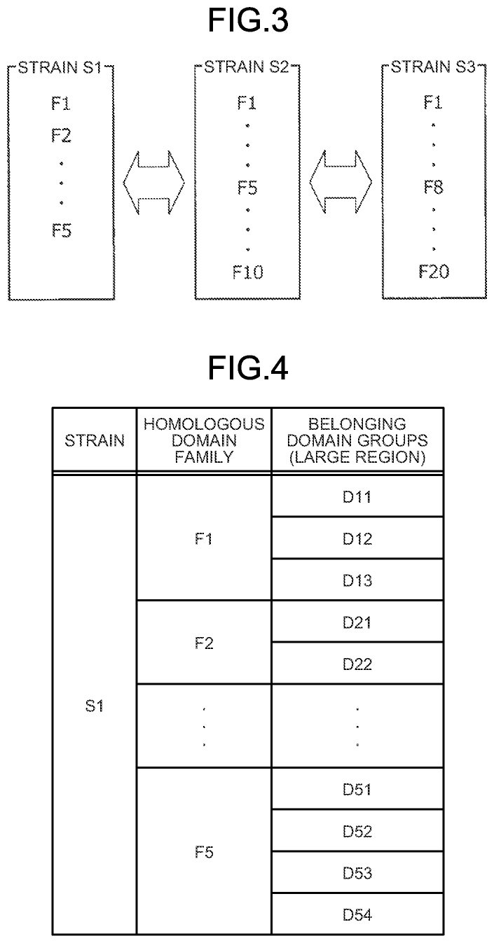

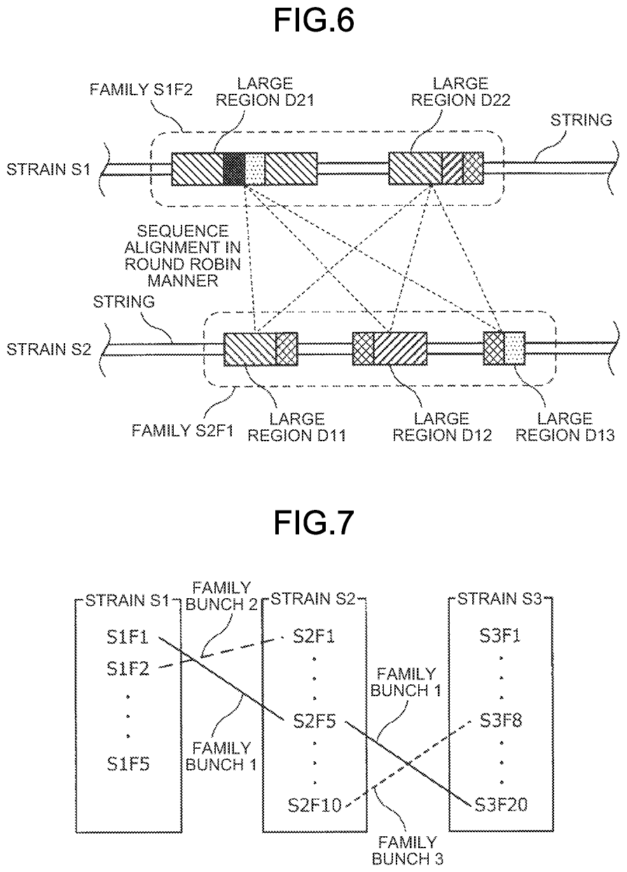

FIG. 2 is a block diagram illustrating an example of the configuration of the homologous domain family bunch classifying unit provided at the information processing apparatus according to the present embodiment. FIG. 3 to FIG. 5 are explanatory diagrams for explaining an example of the homologous domain family information in the present embodiment. FIG. 6 is an explanatory diagram for explaining sequence alignment processing in the present embodiment. FIG. 7 is an explanatory diagram for explaining the homologous domain family bunch in the present embodiment.

As described above, the homologous domain family bunch classifying unit 103 according to the present embodiment analyzes a plurality of pieces of homologous domain family information belonging to a plurality of classification categories, and classifies a group of families “having homology across classification categories (for example, strains of living things (particularly, cells))” for the plurality of pieces of homologous domain family information belonging to the plurality of classification categories, as a “homologous domain family bunch”. As schematically illustrated in FIG. 2, this homologous domain family bunch classifying unit 103 includes, for example, a pre-processing unit 111, a sequence aligning unit 113, a combination extracting unit 115, and a judging unit 117.

The pre-processing unit 111 is realized with, for example, a CPU, a ROM, a RAM, and the like. The pre-processing unit 111 performs various kinds of pre-processing on the homologous domain family information transmitted from the homologous domain family information acquiring unit 101. An example of such pre-processing can include, for example, processing of providing a unique identifier (hereinafter, also referred to as a “global ID”) to a family to globally (that is, across strains which are classification categories) deal with a homologous domain (sequence) existing within a certain strain and a family. The pre-processing unit 111 performs various kinds of pre-processing including the processing of providing an identifier as described above on the homologous domain family information on which attention is focused, and outputs the homologous domain family information subjected to the pre-processing to the sequence aligning unit 113.

The sequence aligning unit 113 is realized with, for example, a CPU, a ROM, a RAM, and the like. The sequence aligning unit 113 performs sequence alignment with all the homologous domain family information on which attention is focused (in other words, in a round robin manner) for content of string pieces included in each of the homologous domain family information (more specifically, the homologous domain family information subjected to the pre-processing) to calculate a degree of similarity among the content of the string pieces.

Here, a method for calculating a degree of similarity among the content of the string pieces is not limited, and various kinds of degrees of similarity (or degrees of difference) may be calculated using a pattern matching technique, or various kinds of degrees of similarity (or degrees of difference) may be calculated using a machine learning technique.

The sequence aligning unit 113 may calculate, for example, a degree of similarity or a score indicating a degree of similarity between two pieces of string data as a feature amount indicating a degree of similarity using the pattern matching technique. Alternatively, the sequence aligning unit 113 may calculate various kinds of distance scales indicating a degree of difference between two pieces of string data as a feature amount indicating a degree of difference. For example, in calculation of a distance scale between two pieces of string data, the sequence aligning unit 113 can calculate a known distance scale including various kinds of distance scales such as a Hamming distance, a Manhattan distance, a Levenshtein distance and a Smith-Waterman distance, a distance scale obtained by combining these distance scales with entropy and N-gram, and the like.

The sequence aligning unit 113 performs sequence alignment with all the homologous domain family information on which attention is focused in a round robin manner to calculate a degree of similarity among content of string pieces, and outputs a feature amount indicating the calculated degree of similarity, and information indicating a combination of the content of the string pieces providing the feature amount to the combination extracting unit 115.

The combination extracting unit 115 is realized with, for example, a CPU, a ROM, a RAM, and the like. The combination extracting unit 115 extracts a combination of families including one or more combinations of content of string pieces for which the degree of similarity is equal to or higher than a predetermined threshold, as a family group having homology. By this means, the combination of families which are considered to have homology is to be extracted from combinations of all the homologous domain family information on which attention is focused. The combination extracting unit 115 outputs information regarding the extracted family group having homology to the judging unit 117.

The judging unit 117 is realized with, for example, a CPU, a ROM, a RAM, and the like. The judging unit 117 judges the content of the string pieces constituting the extracted family group having homology as belonging to the same homologous domain family bunch. By this means, a plurality of pieces of homologous domain family information on which attention is focused are classified into homologous domain family bunches (global bunches) constituted with family groups having homology.

Here, there can be a case where a family which has already belonged to a certain homologous domain family bunch also belongs to another homologous domain family bunch in classification of family groups having homology into the homologous domain family bunches. In such a case, the judging unit 117 preferably merges the homologous domain family bunches to which families belong in common. Such merging processing enables the homologous domain family information on which attention is focused to be classified into the homologous domain family bunch more accurately.

The configuration of the homologous domain family bunch classifying unit 103 according to the present embodiment has been described in detail above with reference to FIG. 2.

[Specific Examples of Homologous Domain Family Bunch Classification Processing]

Subsequently, processing of classifying homologous domain family bunches according to the present embodiment will be specifically described with reference to FIG. 3 to FIG. 7.

In the following description, as schematically illustrated in FIG. 3, attention is focused on a case where processing of classifying homologous domain family bunches is performed using three types of homologous domain family information which is obtained by respectively applying the homologous domain phylogeny analysis method disclosed in Patent Literature 1 to a group of living things (for example, cells, or the like) to which three types of strains of a strain S1, a strain S2 and a strain S3 belong.

As schematically illustrated in FIG. 3, it is assumed that the strain S1 has 5 families (homologous domain families) of F1 to F5, the strain S2 has 10 families (homologous domain families) of F1 to F10, and the strain S3 has 20 families (homologous domain families) of F1 to F20.

In this case, as schematically illustrated in FIG. 4, for example, in the homologous domain family information regarding the strain S1, information regarding the strain, information regarding the families (homologous domain families) of the strain, and information regarding content of homologous string pieces belonging to the respective families are associated with one another.

Here, examples of the “content of homologous string pieces belonging to the families” included in the homologous domain family information can include domain groups belonging to the homologous domain families, and examples of the domain groups can include, for example, a “large region (domain) D” disclosed in the above-described Patent Literature 1. In the homologous domain phylogeny analysis method disclosed in the above-described Patent Literature 1, a region including a homologous section and margin sections which have a predetermined length and which are added to both ends of the homologous section are set as a small region (region), a small region which is included in none of the other small regions is set as a middle region (ceiling), and a group of the overlapped middle regions is set as a large region (domain). Further, various kinds of regions are collectively referred to as a regional group.

Further, in the homologous domain phylogeny analysis method disclosed in the above-described Patent Literature 1, a combined group of a homological group B={Bᵢ} and a regional group X={Xⱼ} is set as one family, and a combined group of the homological group B={Bᵢ} and a large region D={Dⱼ} is set as a large family. In other words, in the present embodiment, a homologous domain family Fi illustrated in FIG. 3, and the like, corresponds to the large family in the above-described Patent Literature 1.

In a case of the homologous domain family information of the strain S1 illustrated in FIG. 4, it is indicated that the five homologous domain families of F1 to F5 exist in the strain S1, and three types of domain groups (large regions) of D11 to D13 are indicated in the family F1 as an example of the “content of the homologous string pieces belonging to the families”. Such association of information is similarly indicated for the families F2 to F5. Further, the strain S2 and the strain S3 also include information having a configuration similar to that illustrated in FIG. 4.

Upon input of the three types of homologous domain family information as described above at the homologous domain family bunch classifying unit 103 according to the present embodiment, the pre-processing unit 111 of the homologous domain family bunch classifying unit 103 defines a homologous domain family bunch group {Bi} (where i is an integer for identifying the homologous domain family bunch group) first as (step 1). Here, in a case where processing of classifying the homologous domain family bunch group is performed for the first time, the homologous domain family bunch group {Bi} is defined as an empty set.

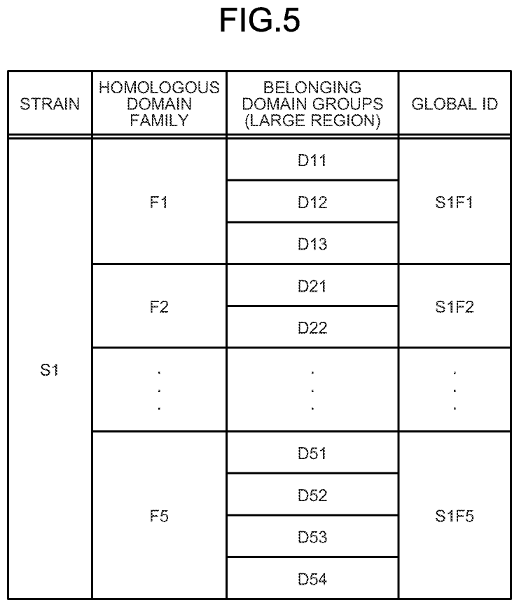

Then, the pre-processing unit 111 provides global IDs to respective families included in the homologous domain family information of each strain as (step 2). Such a global ID is not limited, but, for example, “SiFj” which is obtained by linking “Si” which is an identifier provided to a strain with “Fj” which is an identifier provided to a homologous domain family can be dealt as the global ID. As a result of such a global ID being provided, for example, as schematically illustrated in FIG. 5, global IDs are provided to all families included in the homologous domain family information.

Subsequently, the pre-processing unit 111 confirms whether the homologous domain family on which attention is focused belongs to any homologous domain family bunch {Bi} so far as (step 3). In a case where the homologous domain family does not belong to any homologous domain family bunch, the pre-processing unit 111 newly defines a homologous domain family bunch {Bi} including the homologous domain family on which attention is focused as an element.

In the following description, description will be continued assuming that a homologous domain family bunch B1 including a homologous domain family S1F1 as an element is newly defined, and, in a similar manner, a homologous domain family bunch B2 including a homologous domain family S1F2 as an element and a homologous domain family bunch B6 including a homologous domain family S2F1 as an element are defined.

After the pre-processing as described above is performed, the sequence aligning unit 113 and the combination extracting unit 115 perform the following series of processing as (step 4). In other words, the sequence aligning unit 113 calculates a feature amount indicating a degree of similarity by performing alignment between a domain group (large region D) of a certain homologous domain family of a certain strain and a domain group (large region D) of a certain homologous domain family of another strain in a round robin manner. Then, the combination extracting unit 115 extracts a combination of families including one or more feature amounts indicating homology from combinations of all domain groups obtained through alignment in a round robin manner, as a family group having homology on the basis of the calculated feature amount indicating the degree of similarity.

For example, as schematically illustrated in FIG. 6, a case will be considered where alignment is performed between a domain group {S1F2Di} of the homologous domain family S1F2 and a domain group {S2F1Dj} of the homologous domain family S2F1. It is assumed in this case that two large regions D21 and D22 belong to the homologous domain family S1F2, and three large regions D11, D12 and D13 belong to the homologous domain family S2F1. In this case, the sequence aligning unit 113 calculates feature amounts indicating six types of degrees of similarity by performing alignment in a round robin manner. Here, if at least one combination for which homology is found is included among six types of combinations of {S1F2D21}×{S2F1D11}, {S1D2D22}×{S2F1D11}, {S1F2D21}×{S2F1D12}, {S1D2D22}×{S2F1D12}, {S1F2D21}×{S2F1D13} and {S1D2D22}×{S2F1D13}, the combination extracting unit 115 determines that the homologous domain family S1F2 and the homologous domain family S2F1 have homology and extracts a combination of the homologous domain family S1F2 and the homologous domain family S2F1.

Then, the judging unit 117 judges that content (that is, the large region D) of the string pieces which respectively constitute the homologous domain families extracted by the combination extracting unit 115 belong to the same homologous domain family bunch as (step 5). By this means, the homologous domain families for which homology is found belong to the same homologous domain family bunch.

Note that, in a case where the respective homologous domain families have belonged to another homologous domain family bunches so far, the judging unit 117 merges these homologous domain family bunches. For example, in a case of the above description which assumes that the homologous domain family S1F1 belongs to the homologous domain family bunch B2, and the homologous domain family S2F1 belongs to the homologous domain family bunch B6, the homologous domain family bunch B6 is merged to the homologous domain family bunch B2, so that the homologous domain family S1F2 and the homologous domain family S2F1 belong to the homologous domain family bunch B2 as domain groups.

Through the processing as described above, for example, as schematically illustrated in FIG. 7, all the families included in the three types of homologous domain family information are classified into a plurality of homologous domain family bunches and a database of the homologous domain family bunch group is updated.

Note that, in a case where the processing of classifying homologous domain family bunches proceeds with a group of living things in which another strain S4 is newly added to a group of living things including the strain S1 to the strain S3 illustrated in FIG. 3, the homologous domain family bunch classifying unit 103 performs the above-described processing from (step 2) to (step 5) on the basis of the database of the homologous domain family bunch group updated through the processing as described above. By this means, a database of the homologous domain family bunch group to which information regarding the strain S4 is newly added is constructed.

In this manner, the processing of classifying homologous domain family bunches according to the present embodiment classifies respective homologous domain families which include one or more combinations of domain groups for which homology is found and which constitute the homologous domain families which are generic concept including these domain groups, into the same homologous domain family bunch. More specifically, it can be considered that the processing of classifying homologous domain family bunches according to the present embodiment performs loose filtering on a plurality of pieces of homologous domain family information on which attention is focused. The homologous domain families including at least one combination of domain groups for which homology is found are classified into the same homologous domain family bunch, so that the processing of classifying homologous domain family bunches according to the present embodiment can extract a combination having homology using various elements of the domain group (large region), for example, as illustrated in FIG. 6 as keys.

The processing of classifying homologous domain family bunches according to the present embodiment has been specifically described with reference to FIG. 3 to FIG. 7.

Heretofore, an example of the function of the information processing apparatus 10 according to the present embodiment has been described. Each of the structural elements described above may be configured using a general-purpose material or circuit, or may be implemented by hardware dedicated to the function of each structural element. Further, the function of each structural element may be carried out by a CPU or the like. Accordingly, the configuration to be used can be changed as appropriate according to the technical level at the time of carrying out the present embodiment.

Note that it is possible to develop a computer program for realizing the respective functions of the information processing apparatus according to the present embodiment as described above, and implement the computer program in a personal computer or the like. In addition, a computer-readable recording medium storing such a computer program may also be provided. The recording medium may be a magnetic disk, an optical disc, a magneto-optical disk, flash memory, or the like, for example. Furthermore, the above computer program may also be delivered via a network, for example, without using a recording medium.

<Flow of Information Processing Method>

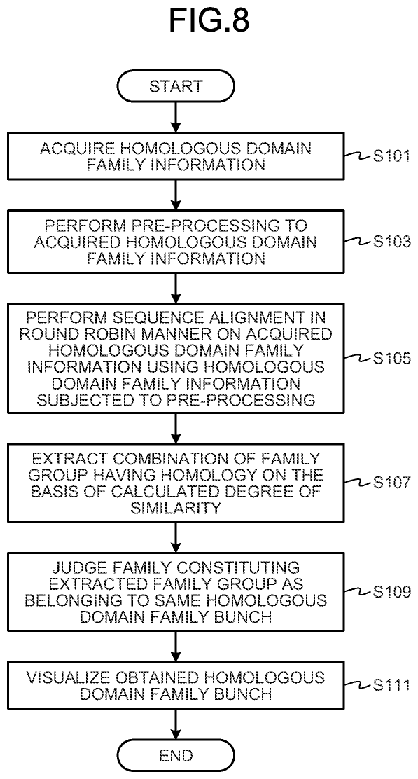

An example of flow of an information processing method to be performed at the information processing apparatus 10 according to the present embodiment as described above will be briefly described next with reference to FIG. 8. FIG. 8 is a flowchart illustrating an example of flow of the information processing method according to the present embodiment.

In the information processing method according to the present embodiment, first, the homologous domain family information acquiring unit 101 acquires homologous domain family information on which attention is focused (step S101), and outputs the acquired homologous domain family information to the homologous domain family bunch classifying unit 103.

The pre-processing unit 111 of the homologous domain family bunch classifying unit 103 which acquires the homologous domain family information performs various kinds of pre-processing such as, for example, processing of providing a global ID to the acquired homologous domain family information (step S103). The homologous domain family information subjected to the pre-processing is output to the sequence aligning unit 113.

The sequence aligning unit 113 performs sequence alignment in a round robin manner on content of string pieces included in the respective pieces of homologous domain family information using the homologous domain family information subjected to the pre-processing (step S105) and calculates a degree of similarity among the content of the string pieces (for example, a large region D as an example of domain groups constituting a family). After the sequence aligning unit 113 calculates the degree of similarity among the content of the string pieces, the sequence aligning unit 113 outputs a calculation result of the degree of similarity to the combination extracting unit 115.

The combination extracting unit 115 extracts a combination of families including one or more combinations of content of string pieces for which the degree of similarity is equal to or higher than a predetermined threshold, as a family group having homology (step S107). By this means, the combination of families which are considered to have homology is extracted from combinations of all the homologous domain family information on which attention is focused.

Subsequently, the judging unit 117 judges the content of the string pieces constituting the extracted family group having homology as belonging to the same homologous domain family bunch (step S109). By this means, a plurality of pieces of homologous domain family information on which attention is focused are classified into homologous domain family bunches constituted with family groups having homology.

Further, in the information processing method according to the present embodiment, the visualizing unit 105 may visualize the obtained homologous domain family bunch on the basis of relationship of belonging of the homologous domain family bunch (step S111). By this means, it becomes possible to easily grasp relationship of sharing of the homologous domain family bunch among a plurality of classification categories (for example, “strains” such as cells).

An example of the flow of the information processing method to be performed at the information processing apparatus 10 according to the present embodiment has been briefly described above with reference to FIG. 8.

<Modified Example of Information Processing Apparatus>

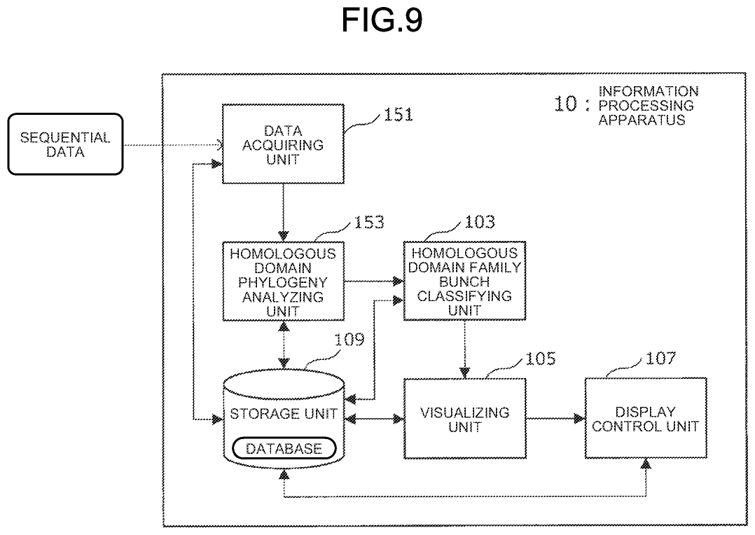

In the above description, the information processing apparatus 10 according to the present embodiment acquires the homologous domain family information which is an analysis result of the homologous domain phylogeny analysis method from outside and performs processing of classifying homologous domain family bunches on the basis of the acquired homologous domain family information. However, the information processing apparatus 10 itself may have a homologous domain phylogeny analyzing function of performing the homologous domain phylogeny analysis method on sequential data (sequential data including characters, signs and numerical values) on which attention is focused. A modified example of the information processing apparatus 10 according to the present embodiment will be briefly described below with reference to FIG. 9. FIG. 9 is a block diagram illustrating an example of a configuration of the modified example of the information processing apparatus according to the present embodiment.

As schematically illustrated in FIG. 9, the information processing apparatus 10 according to the present modified example includes a data acquiring unit 151 and a homologous domain phylogeny analyzing unit 153 in place of the homologous domain family information acquiring unit 101 illustrated in FIG. 1. The homologous domain family bunch classifying unit 103, the visualizing unit 105, the display control unit 107 and the storage unit 109 in the present modified example have similar functions and provide similar effects as those illustrated in FIG. 1, and thus, description will be omitted below.

The data acquiring unit 151 is realized with, for example, a CPU, a ROM, a RAM, an input device, a communication device, and the like. The data acquiring unit 151 acquires sequential data (for example, string data) which is a target of the homologous domain phylogeny analysis method as described above.

The data acquiring unit 151 may acquire the above-described sequential data (for example, string data) from various kinds of apparatuses connected via a network such as the Internet and a home network or may acquire the sequential data from various kinds of apparatuses directly connected to the information processing apparatus 10 in a wired or wireless manner. Alternatively, the data acquiring unit 151 may set data directly input by a user to the information processing apparatus 10 via various kinds of input devices such as a keyboard and a touch panel, as the sequential data.

The data acquiring unit 151 outputs the acquired sequential data (for example, string data) to the homologous domain phylogeny analyzing unit 153 which will be described later. Further, the data acquiring unit 151 may store the acquired sequential data in the storage unit 109, or the like, in association with time information regarding date, time, and the like, at which the sequential data is acquired.

The homologous domain phylogeny analyzing unit 153 is realized with, for example, a CPU, a ROM, a RAM, a communication device, and the like. The homologous domain phylogeny analyzing unit 153 is a processing unit which performs analysis using the homologous domain phylogeny analysis method developed by the present inventor to generate homologous domain family information. More specifically, the homologous domain phylogeny analyzing unit 153 analyzes string data which is an example of the sequential data output from the data acquiring unit 151 to extract homologous string pieces within a string indicated by the string data, and performs phylogeny analysis on the extracted homologous string pieces to generate homologous domain family information.

Here, specific processing of the homologous domain phylogeny analysis method performed at the homologous domain phylogeny analyzing unit 153 is similar to that performed at the homologous domain phylogeny analyzing unit in the above-described Patent Literature 1, and thus, detailed description will be omitted below.

Note that, in a case where the data acquiring unit 151 in the present modified example acquires sequential data to be analyzed from various kinds of server, or the like, connected via a network, it is also possible to automate acquisition processing of sequential data so that the data acquiring unit 151 can automatically acquire sequential data from a desired database of sequential data. It becomes thereby possible to implement homologous domain phylogeny analysis on the acquired sequential data more efficiently.

Further, the sequence alignment processing to be performed at the homologous domain phylogeny analyzing unit 153 may utilize a unique program constructed with unique algorithm or may utilize a known sequence alignment software typified by Blastn, or the like. In a case where the known sequence alignment software is utilized, if possible, the sequence alignment software is preferably incorporated into a system of the information processing apparatus 10, so that a series of processing is implemented within the same apparatus. Further, in a case where the known sequence alignment software is utilized, optimal values of various kinds of parameters are preferably set in advance so that cost for inserting a gap in alignment becomes the highest (in other words, so as to insert as few gaps as possible) for various kinds of parameters of the sequence alignment software. By this means, it becomes possible to implement homologous domain phylogeny analysis more efficiently.

The information processing apparatus 10 according to the present modified example has been briefly described above.

<Hardware Configuration of Information Processing Apparatus 10>

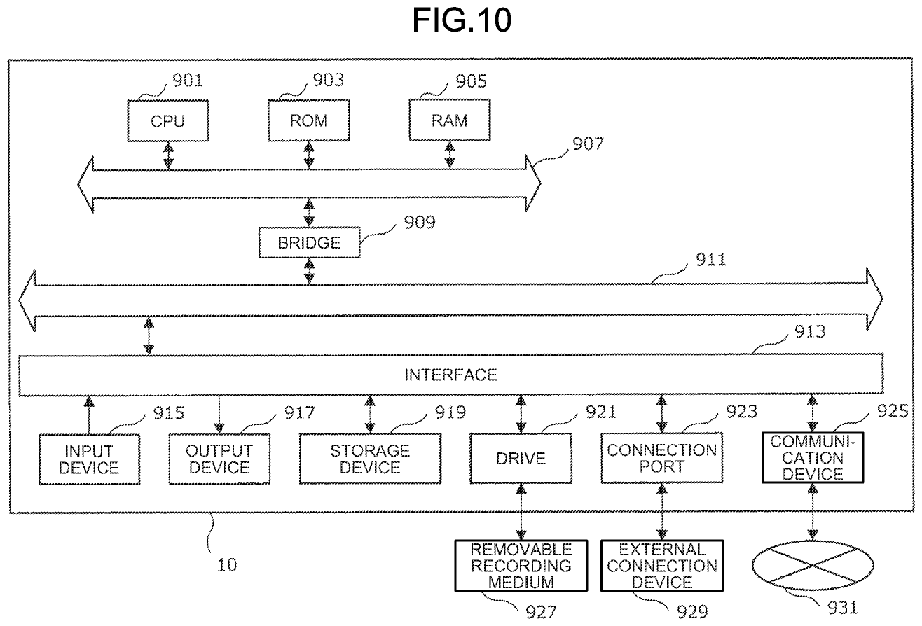

A hardware configuration of the information processing apparatus 10 according to the embodiment of the present disclosure will be described in detail next with reference to FIG. 10. FIG. 10 is a block diagram for explaining the hardware configuration of the information processing apparatus 10 according to the embodiment of the present disclosure.

The information processing apparatus 10 mainly includes a CPU 901, a ROM 903, and a RAM 905. Furthermore, the information processing apparatus 10 also includes a host bus 907, a bridge 909, an external bus 911, an interface 913, an input device 915, an output device 917, a storage device 919, a drive 921, a connection port 923, and a communication device 925.

The CPU 901 serves as an arithmetic processing device and a control device, and controls the overall operation or a part of the operation of the information processing apparatus 10 according to various programs recorded in the ROM 903, the RAM 905, the storage device 919, or a removable recording medium 927. The ROM 903 stores programs, computational parameters, and the like used by the CPU 901. The RAM 905 temporarily stores programs used by the CPU 901, parameters that change as appropriate during the execution of the programs, and the like. These are connected with each other via the host bus 907 including an internal bus such as a CPU bus.

The host bus 907 is connected to the external bus 911 such as a Peripheral Component Interconnect/Interface (PCI) bus via the bridge 909.

The input device 915 is an operation mechanism operated by a user, such as a mouse, a keyboard, a touch panel, buttons, a switch, or a lever, for example. Also, the input device 915 may be a remote control mechanism (a so-called remote control) using, for example, infrared light or other radio waves, or may be an external connection device 929 such as a mobile phone or a PDA conforming to the operation of the information processing apparatus 10. Furthermore, the input device 915 generates an input signal on the basis of, for example, information which is input by a user with the above operation mechanism, and includes an input control circuit for outputting the input signal to the CPU 901. The user of the information processing apparatus 10 can input various data to the information processing apparatus 10 and can instruct the information processing apparatus 10 to perform processing by operating the input device 915.

The output device 917 includes a device capable of visually or audibly notifying a user of acquired information. Examples of such a device include display devices such as a CRT display device, a liquid crystal display device, a plasma display device, an EL display device, and lamps, audio output devices such as a speaker and a headphone, a printer, a mobile phone, a facsimile, and the like. For example, the output device 917 outputs a result obtained by various processes performed by the information processing apparatus 10. More specifically, the display device displays, in the form of texts or images, a result obtained by various processes performed by the information processing apparatus 10. On the other hand, the audio output device converts an audio signal including reproduced audio data, sound data, and the like into an analog signal, and outputs the analog signal.

The storage device 919 is a device for storing data configured as an example of a storage unit of the information processing apparatus 10. The storage device 919 is configured from, for example, a magnetic storage device such as a Hard Disk Drive (HDD), a semiconductor storage device, an optical storage device, or a magneto-optical storage device. This storage device 919 stores programs to be executed by the CPU 901, various data, and various data obtained externally, and the like.

The drive 921 is a reader/writer for recording medium, and is embedded in the information processing apparatus 10 or attached externally thereto. The drive 921 reads information recorded in the attached removable recording medium 927 such as a magnetic disk, an optical disc, a magneto-optical disk, or a semiconductor memory, and outputs the read information to the RAM 905. Furthermore, the drive 921 can write record in the attached removable recording medium 927 such as a magnetic disk, an optical disc, a magneto-optical disk, or a semiconductor memory. The removable recording medium 927 is, for example, a DVD medium, an HD-DVD medium, a Blu-ray (registered trademark) medium, or the like. In addition, the removable recording medium 927 may be a CompactFlash (CF; registered trademark), a flash memory, a Secure Digital Memory Card (SD memory card), or the like. Alternatively, the removable recording medium 927 may be, for example, an Integrated Circuit Card (IC card) equipped with a non-contact IC chip, an electronic appliance, or the like.

The connection port 923 is a port for allowing devices to directly connect to the information processing apparatus 10. Examples of the connection port 923 include a Universal Serial Bus (USB) port, an IEEE1394 port, a Small Computer System Interface (SCSI) port, and the like. Other examples of the connection port 923 include an RS-232C port, an optical audio terminal, a High-Definition Multimedia Interface (HDMI) (registered trademark) port, and the like. By the external connection device 929 connecting to this connection port 923, the information processing apparatus 10 directly obtains various types of data from the external connection device 929 and provides various types of data to the external connection device 929.

The communication device 925 is a communication interface including, for example, a communication device for connecting to a communication network 931 or the like. The communication device 925 is, for example, a wired or wireless Local Area Network (LAN), Bluetooth (registered trademark), a communication card for Wireless USB (WUSB), or the like. Alternatively, the communication device 925 may be a router for optical communication, a router for Asymmetric Digital Subscriber Line (ADSL), a modem for various communications, or the like. This communication device 925 can transmit and receive signals and the like in accordance with a predetermined protocol such as TCP/IP on the Internet and with other communication devices, for example. In addition, the communication network 931 connected to the communication device 925 includes a network and the like, which is connected via wire or wirelessly, and may be, for example, the Internet, a home LAN, infrared communication, radio wave communication, satellite communication, or the like.

Heretofore, an example of the hardware configuration capable of realizing the functions of the information processing apparatus 10 according to the embodiment of the present disclosure has been shown. Each of the structural elements described above may be configured using a general-purpose material, or may be implemented by hardware dedicated to the function of each structural element. Accordingly, the hardware configuration to be used can be changed as appropriate according to the technical level at the time of carrying out the present embodiment.

### EXAMPLES

Typically, genetic information of genomes is not fixed from birth until death of living things. For example, in a case where a human gets infected with a virus, the virus causes genetic information of DNA or RNA of the virus to infiltrate into a cell of the human who is a host, and inserts part or all of the genetic information into genomes of the host so as to transfer the genetic information to posterity as well as staying for many decades. Different living things (and parasites) have a way to adapt to change of an environment by exchanging genetic information upon meeting. It is known that most of pathogenic bacteria (bacteria which adversely affect a human body) also have a peculiar DNA region or RNA region called a pathological island, which is not possessed by other bacteria and which is to be inserted into a host.

Most of the homologous domains on which attention is focused in the homologous domain phylogeny analysis method developed by the present inventor and the method for classifying homologous domain family bunches proposed in the present disclosure are mobile elements which transfer between genomes between different living things or within one living thing and provide plasticity to the genomes as part of survival strategy in accordance with an environment. Such homologous domains include homologous domains which are left by other parasites, pathogenic organisms, viruses or bacteriophages at a host as well as homologous domains which transfer between different genome molecules (chromosomes and plasmids) within one living thing.

Genome sequence information of many types of living things in addition to a human have been deciphered since 1990. Further, in recent years, as a result of existence of resident microbiota in the body including bacteria in intestines becoming obvious, it is guessed that these resident microbiota in the body play various roles in defense, immunity, feeling, brain activity, and the like, and it has been recognized that a life of a human is sustained by a symbiont with the resident microbiota in the body. It is expected to elucidate relationship among a human body, resident microbiota in the body and pathogenic microorganisms (such as mold, bacteria and viruses) from outside and a molecular mechanism of infection and parasitism of them for the purpose of health maintenance, anti-aging, prevention of lifestyle diseases and autoimmune diseases.

It becomes possible to grasp encounter and specialization among different living things through homologous domain phylogeny by tracking classification and relationship of homologous domain phylogeny in microbial species using the homologous domain phylogeny analysis method. Further, there is a possibility that homologous domain phylogeny which is unique to one living thing is an unknown pathological island. It is expected that comprehensive homologous domain phylogeny analysis of microorganisms will reveal relationship among a human, resident microbiota in the body, pathogenic microorganisms and symbiotic bacteria in food to be ingested as well as being useful for elucidation of biological evolution from birth of the earth, and will be of some help to solution of medical issues that we are facing today.

In terms of this, in examples described below, five strains of Escherichia coli were subjected to the homologous domain phylogeny analysis method and the processing of classifying homologous domain family bunches to visualize relationship of sharing of homologous domain family bunches and analogous relationship among the strains.

The five strains of Escherichia coli on which attention is focused are the following: 1. Escherichia coli IA139 2. Escherichia coli O104:H4 are.2011C-3493 3. Escherichia coli O157:H7 SakaiSakai substr.RIMD059952 4. Escherichia coli O83:H1 str.NRG857C 5. Escherichia coli str.K12substr.MG1655

Escherichia coli K12 indicated in 5 is non-pathogenic Escherichia coli which is normally frequently used for cloning at laboratories. Other four strains indicated in 1 to 4 are pathogenic Escherichia coli.

Escherichia coli IA139 is other name of O7:K1. O7:K1(IA139) is classified into extraintestinal pathogenic Escherichia coli (EE×PEC) which causes unitary tract infection, meningitis, sepsis, and the like, and was isolated from urine of patients of unitary tract infection at France in the 1980s.

Escherichia coli O104:H4, Escherichia coli O157:H7, and Escherichia coli O83:H1 are respectively classified into enterohemorrhagic Eschericia coli (EHEC), and are Escherichia coli which produces a strong toxin called a verotoxin. While Escherichia coli is characterized by O antigen of antiserum against a verotoxin, there is also Escherichia coli which does not produce a verotoxin in a case where Escherichia coli has the O antigen, but has different H antigen. Therefore, enterohemorrhagic Escherichia coli is expressed with combination of O antigen and H antigen, like O157:H7.

Escherichia coli O104:H4 are.2011C-3493 was isolated from patients of a large-scale mass food poisoning occurred in France and Germany in 2011, Escherichia coli O157:H7 Sakai was isolated from patients of a mass food poisoning occurred at Sakai-shi, and Escherichia coli O83:H1 was isolated from ileums of patients with Crohn's disease. It is known that, among these Escherichia coli, particularly, O157:H1 produces an extremely poisonous verotoxin which is fatal.

In a medical research field of enterohemorrhagic infection, it is desired to understand a mechanism of its pathogenicity and develop treatments and prevention measures, and the pathogenicity has been searched for using a method of comparing all genes by comparing a whole genome sequence of pathogenic Escherichia coli and non-pathogenic Escherichia coli. As a result, it has been already reported that there is few difference in genetic information on chromosomes between pathogenic Escherichia coli and non-pathogenic Escherichia coli, and pathogenicity indicated by Escherichia coli is caused by not genes on chromosomes but genes on plasmids.

In the examples described below, the above-described five strains of Escherichia coli were subjected to the homologous domain phylogeny analysis and classification of homologous domain family bunches using the information processing apparatus 10 having a configuration illustrated in FIG. 9. At the information processing apparatus 10 which is used, genome data of five strains of Escherichia coli were automatically acquired from National Center for Biotechnology Information (NCBI) of U.S.A., and Blastn which is a known sequence alignment software incorporated into an information processing system was used. In this event, optimal values of various kinds of parameters were set in advance so that cost for inserting a gap in alignment becomes the highest (in other words, so as to insert as few gaps as possible) for various kinds of parameters of the sequence alignment software Blastn. Further, it was set at the sequence aligning unit 113 of the homologous domain family bunch classifying unit 103 so that a degree of similarity between two string pieces on which attention is focused is numerically expressed through pattern matching of the string pieces.

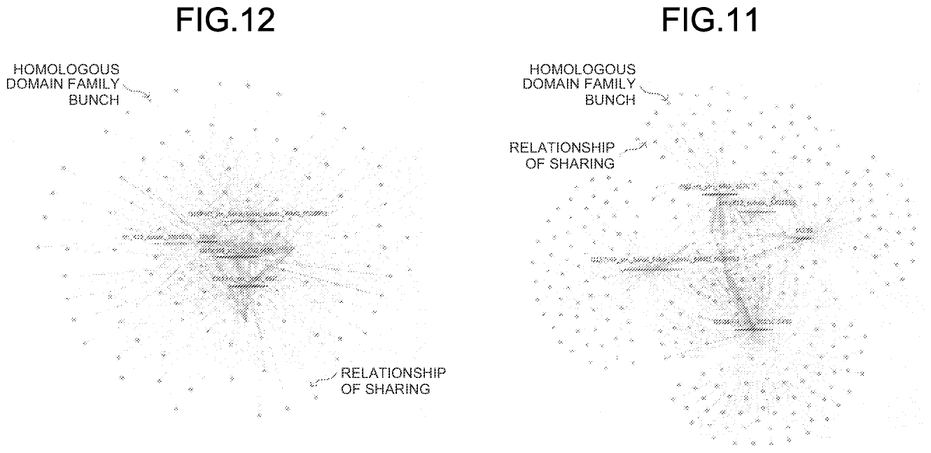

The visualizing unit 105 in the information processing apparatus 10 having the configuration illustrated in FIG. 9 created a drawing indicating relationship of belonging of the homologous domain family bunches to respective strains, which was obtained through the homologous domain phylogeny analysis and the classification of homologous domain family bunches as described above, using sfdp commands of Graphviz which is a known graph structure drawing software. The obtained result is illustrated in FIG. 11. FIG. 11 illustrates the obtained homologous domain family bunches with circular dots, illustrates the strains with ellipses, and illustrates the relationship of belonging of the homologous domain family bunches to the strains with curves. Note that, to create a distribution map illustrated in FIG. 11, the visualizing unit 105 is set so that the homologous domain family bunches are distributed to the whole drawing plane, and the distribution map illustrated in FIG. 11 does not have concept such as coordinate axes.

In FIG. 11, thicker curves indicate sharing of the homologous domain family bunches by more strains. It can be known from FIG. 11 that a lot of homologous domain family bunches liked with only the strain exist outside the respective strains. These homologous domain family bunches are local homologous domain family bunches which emerge only in a single strain. A distribution map illustrated in FIG. 12 does not indicate local homologous domain family bunches which emerge only in a single strain and indicates only homologous domain family bunches shared by a plurality of strains.

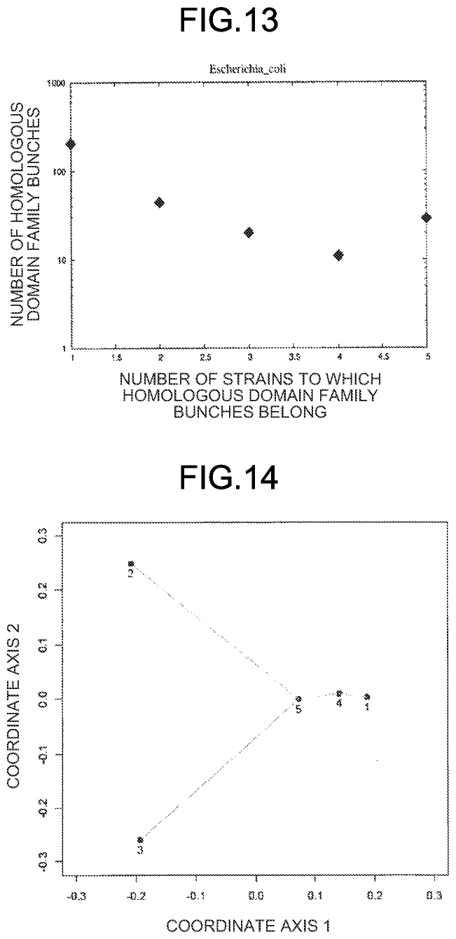

FIG. 13 illustrates relationship between the number of homologous domain families and the number of belonging strains (that is, a histogram regarding the number of strains to which the respective homologous domain family bunches belong) for the homologous domain family bunches found in the five strains of Escherichia coli. FIG. 13 indicates the number of strains to which the homologous domain family bunches belong on a horizontal axis and indicates the number on a vertical axis. The total number of the homologous domain family bunches found in the five strains of Escherichia coli was 309. As can be clear from a plot located at a value of 1 on the horizontal axis in FIG. 13, most of the homologous domain family bunches are local homologous domain family bunches which emerge only in a single strain. Meanwhile, as can be clear from a plot located at a value of 5 on the horizontal axis in FIG. 13, the number of the homologous domain family bunches which emerge in common to all five strains was 29.

It became apparent from part of 29 homologous domain family bunches which emerge in common to all five strains that the homologous domain family bunch B6 is derived from transposons because it contains transposase genes. Further, it became apparent that the homologous domain family bunch B8 is related to RNA because it contains genes such as 16SribosomalRNA, 23SribosomalRNA, tRNA-Ile and tRNA-Asp. Further, it became apparent that the homologous domain family bunch B13, which includes a pyruvate dehydrogenase gene, is involved with control of a TCA cycle which is the center of a biochemical pathway. Further, it became apparent that the homologous domain family bunch B24, which includes a transcriptional regulator HU subunit beta (hupB) gene, and in which HU protein has characteristics in common with histone, is involved with a broad range of gene expression control.

Meanwhile, several homologous domain family bunches which belong to four strains of pathogenic Escherichia coli (IAI39, O104:H4, 0157:H7 and O82:H1) or part of the four strains, but do not belong to non-pathogenic Escherichia coli K12 were found through the above-described test.

(A) Two homologous domain family bunches of B2 and B15 which belong to three strains of O104:H4, 0157:H7 and O82:H1, but do not belong to IAI39 and K12 were found.

Three homologous domain families (F2, F3 and F4) of an extremely poisonous strain O157:H7 is linked with the homologous domain family bunch B2, in which two domains of F2 include a gene whose function has not been determined, and which is tagged with “hypothetical protein”.

Domains belonging to the homologous domain family bunch B15 included VgrB genes. The VgrB gene is a gene constituting Type VI secretion system, and it is known that the VgrB gene is involved with toxicity and antibacterial properties.

(B) Four homologous domain family bunches of B92, B117, B135 and B139 which belong to three strains of IAI39, O104:H4 and O157:H7, but do not belong to O82:H1 and K12 were found.

All domains belonging to the homologous domain family bunch B92 included nitrate-inducible formate dehydrogenase-Nsubunit alpha genes.

All domains belonging to the homologous domain family bunch B117 included transposaseTnA genes and genes whose functions have not been determined.

All domains belonging to the homologous domain family bunch B135 included adhesin genes or genes whose functions have not been determined.

All domains belonging to the homologous domain family bunch B139 included magnesium/nickel/cobalt transporterCorA genes.

(C) Four homologous domain family bunches of B162, B163, B164 and B173 which belong to IAI39, O83:H1 and K12, but do not belong to two extremely poisonous Enterohemorrhagic Escherichia coli (O104:H4 and O157:H7) were found.

All domains belonging to the homologous domain family bunch B162 included regions displayed as crl(missing), insN(missing), yaiX(missing), yaiT(missing), renD(missing) and nmpC(missing), and cspH genes (members of a CspA family) which encode cold shock protein. It appears that the aforementioned missing genes are derived from insertion elements (IS) and are remaining regions which have lost expression functions.

Likewise, all domains belonging to the homologous domain family bunch B163 included regions displayed as crl (missing), insN (missing), yaiX (missing), yaiT(missing), renD(missing) and nmpC(missing) and cspG genes (members of a CspA family) which encode cold shock protein.

Likewise, all domains belonging to the homologous domain family bunch B164 included regions displayed as crl (missing), insN (missing), yaiX (missing), yaiT(missing), renD(missing) and nmpC(missing) and icd genes derived from e14 prophages (isocitratedegydragenase).

Likewise, all domains belonging to the homologous domain family bunch B173 included regions displayed as lomR(missing), ydbA(missing), ydfH(missing), yoeA(missing), wbbL(missing), gatR(missing) and yej0(missing) in addition to crl (missing), insN (missing), yaiX (missing), yaiT(missing), renD(missing) and nmpC(missing), and “transposase 31 protein” genes derived from transposons.

In this manner, differences in genetic information between pathogenic Escherichia coli and non-pathogenic Escherichia coli, which could not be found using an analysis method which focuses attention on genetic information on chromosomes in related art, could be found through the homologous domain phylogeny analysis method and the processing of classifying homologous domain family bunches according to the present disclosure. In other words, existence of transfer elements derived from transposons and insertion elements could be found as part which differentiates non-pathogenic Escherichia coli from pathogenic Escherichia coli or differentiates Escherichia coli having weak pathogenicity or differentiates Escherichia coli having strong pathogenicity.

Further, 309 homologous domain family bunches in five strains of Escherichia coli which have been found through the homologous domain phylogeny analysis method and the processing of classifying homologous domain family bunches, were analyzed using multi-dimensional scaling analysis (MDS) method. In the multi-dimensional scaling analysis, a function cmdscale of a known statistical processing application R was used. Further, in the analysis, a Manhattan distance D(i, j) between a strain i and a strain j, which is obtained using the following expression (101) was calculated to be made an input value of the multi-dimensional scaling analysis method. In the following expression (101), whether the homologous domain family belonging to the homologous domain family bunch k holds the strain (=1) or does not hold the strain i (=0) is expressed with a bit sequence B(k, i). Note that, in the following expression (101), a parameter NS indicates the number of strains, which is 5 in the present example, and a parameter NB indicates the number of homologous domain family bunches, which is 309 in the present example.

$$D ( i , j ) = \frac{1}{NS \cdot NB} {\sum }_{k = 1}^{NB} \vert B ( k , i ) - B ( k , j ) \vert \qquad Expression ( 101 )$$

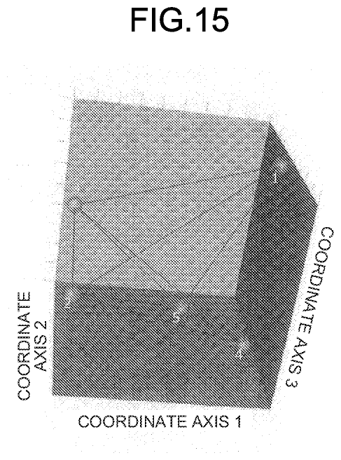

FIG. 14 illustrates a graph obtained by visualizing in two dimensions the analysis result obtained through the multi-dimensional scaling analysis method as described above, and FIG. 15 illustrates a graph obtained by visualizing in three dimensions the analysis result. In FIG. 14, the closest strains are connected to each other with lines, and in FIG. 15, each strain is connected to all other strains with lines. Note that reference numerals 1 to 5 in FIG. 14 and FIG. 15 correspond to the respective strains of the above-described Escherichia coli.

It can be known from FIG. 14 that, while Escherichia coli 1 (IAI39), Escherichia coli 4 (O83:H1) and Escherichia coli 5 (K12) are distributed at positions relatively close to each other within a plane of coordinates, Escherichia coli 2 (O104:H4) and Escherichia coli 3 (O157:H7) which are extremely poisonous pathogenic Escherichia coli are distributed at positions distant from Escherichia coli 1, 4 and 5. Further, it can be known from FIG. 15 that, among Escherichia coli 1, Escherichia coli 4 and Escherichia coli 5 which appear to be located at positions close to each other in FIG. 14, Escherichia coli 1 is distributed at a position distant from Escherichia coli 4 and Escherichia coli 5.

Application of such statistical analysis processing such as multi-dimensional scaling analysis method upon visualization of the homologous domain family bunches enables easy grasp of analogousness and specificity between a strain on which attention is focused and other strains.

As described above using specific examples, the processing of classifying homologous domain family bunches described in the present example can be regarded as a phylogeny analysis method which focuses attention on genetic information which is less shared by living things, and which appears a plurality of times, and excels in extraction of genetic information obtained through horizontal transfer. Such a method can be regarded as a method for estimating whether some kind of interaction has existed in the evolutionary history of living things between living things which share genetic information which is less shared.

Further, most of domains obtained through the homologous domain phylogeny analysis method is genetic information obtained through horizontal transfer, and, as described above, the extracted genetic information is deeply involved with gene expression control. Thus, homologous domain phylogeny analysis method in a broad sense including the processing of classifying homologous domain family bunches as described in the present example, is considered to be useful for, for example, elucidation of a mechanism of acquisition and propagation of toxicity of pathogenic microorganisms including bacteria in intestines and soil bacteria.

As described above, the favorable embodiments of the present disclosure have been described in detail with reference to the accompanying drawings, but the technical scope of the present disclosure is not limited to such examples. It is obvious that persons having ordinary knowledge in the technical field of the present disclosure can conceive various changes and alterations within the scope of the technical idea described in the claims, and it is naturally understood that these changes and alterations belong to the technical scope of the present disclosure.

Furthermore, the effects described in the present specification are merely illustrative or exemplary and are not restrictive. That is, with or in the place of the above effects, the technology according to the present disclosure may achieve other effects that are clear to those skilled in the art from the description of this specification.

Note that the following configuration also belong to the technical scope of the present disclosure.

(1)

An information processing apparatus comprising: a homologous domain family bunch classifying unit configured to classify a group of families having homology across classification categories as a homologous domain family bunch for a plurality of pieces of homologous domain family information belonging to a plurality of the classification categories on the basis of homologous domain family information regarding the families of homologous string pieces and content of the homologous string pieces belonging to the families included in a result of phylogeny analysis by utilizing the result of phylogeny analysis performed on the basis of positions where the homologous string pieces exist and homological relationship of the homologous string pieces while attention is focused on the homologous string pieces within a string for string data representing the string including one or more characters.

(2)

The information processing apparatus according to (1), wherein

the homologous domain family bunch classifying unit comprises:

a sequence aligning unit configured to perform sequence alignment with all the homologous domain family information on which attention is focused for content of the string pieces included in each of the homologous domain family information, to calculate a degree of similarity among the content of the string pieces;

a combination extracting unit configured to extract a combination of the families which include one or more combinations of content of string pieces for which the degree of similarity is equal to or higher than a predetermined threshold, as a family group having homology; and

a judging unit configured to judge the content of the string pieces constituting the extracted family group having homology as belonging to the same homologous domain family bunch.

(3)

The information processing apparatus according to (1) or (2), further comprising: a visualizing unit configured to visualize a plurality of the classified homologous domain family bunches on the basis of relationship of belonging of the homologous domain family bunches to the classification categories.

(4)

The information processing apparatus according to (3), wherein the visualizing unit visualizes analogous relationship of the classification categories by further performing at least one of principal component analysis on the homologous domain family bunches belonging to the classification categories or multi-dimensional scaling, on a plurality of the classified homologous domain family bunches.

(5)

The information processing apparatus according to any one of (1) to (4), wherein

the string data is string data representing genetic information of living things, and

the classification categories are strains of the living thing having the string data.

(6)

An information processing method comprising: classifying a group of families having homology across classification categories as a homologous domain family bunch for a plurality of pieces of homologous domain family information belonging to a plurality of the classification categories on the basis of homologous domain family information regarding the families of homologous string pieces and content of the homologous string pieces belonging to the families included in a result of phylogeny analysis by utilizing the result of phylogeny analysis performed on the basis of positions where the homologous string pieces exist and homological relationship of the homologous string pieces while attention is focused on the homologous string pieces within a string for string data representing the string including one or more characters.

(7)

A program for causing a computer to realize a homologous domain family bunch classifying function of classifying a group of families having homology across classification categories as a homologous domain family bunch for a plurality of pieces of homologous domain family information belonging to a plurality of the classification categories on the basis of homologous domain family information regarding the families of homologous string pieces and content of the homologous string pieces belonging to the families included in a result of phylogeny analysis by utilizing the result of phylogeny analysis performed on the basis of positions where the homologous string pieces exist and homological relationship of the homologous string pieces while attention is focused on the homologous string pieces within a string for string data representing the string including one or more characters.

### REFERENCE SIGNS LIST

10 INFORMATION PROCESSING APPARATUS
101 HOMOLOGOUS DOMAIN FAMILY INFORMATION ACQUIRING UNIT
103 HOMOLOGOUS DOMAIN FAMILY BUNCH CLASSIFYING UNIT
105 VISUALIZING UNIT
107 DISPLAY CONTROL UNIT
109 STORAGE UNIT
111 PRE-PROCESSING UNIT
113 SEQUENCE ALIGNING UNIT
115 COMBINATION EXTRACTING UNIT
117 JUDGING UNIT
151 DATA ACQUIRING UNIT
153 HOMOLOGOUS DOMAIN PHYLOGENY ANALYZING UNIT

1. An information processing apparatus comprising: a homologous domain family bunch classifying unit configured to classify a group of families having homology across classification categories as a homologous domain family bunch for a plurality of pieces of homologous domain family information belonging to a plurality of the classification categories on the basis of homologous domain family information regarding the families of homologous string pieces and content of the homologous string pieces belonging to the families included in a result of phylogeny analysis by utilizing the result of phylogeny analysis performed on the basis of positions where the homologous string pieces exist and homological relationship of the homologous string pieces while attention is focused on the homologous string pieces within a string for string data representing the string including one or more characters.

2. The information processing apparatus according to claim 1, wherein
the homologous domain family bunch classifying unit comprises:
a sequence aligning unit configured to perform sequence alignment with all the homologous domain family information on which attention is focused for content of the string pieces included in each of the homologous domain family information, to calculate a degree of similarity among the content of the string pieces;
a combination extracting unit configured to extract a combination of the families which include one or more combinations of content of string pieces for which the degree of similarity is equal to or higher than a predetermined threshold, as a family group having homology; and
a judging unit configured to judge the content of the string pieces constituting the extracted family group having homology as belonging to the same homologous domain family bunch.

3. The information processing apparatus according to claim 1, further comprising: a visualizing unit configured to visualize a plurality of the classified homologous domain family bunches on the basis of relationship of belonging of the homologous domain family bunches to the classification categories.

4. The information processing apparatus according to claim 3, wherein the visualizing unit visualizes analogous relationship of the classification categories by further performing at least one of principal component analysis on the homologous domain family bunches belonging to the classification categories or multi-dimensional scaling, on a plurality of the classified homologous domain family bunches.

5. The information processing apparatus according to claim 1, wherein
the string data is string data representing genetic information of living things, and
the classification categories are strains of the living thing having the string data.

6. An information processing method comprising: classifying a group of families having homology across classification categories as a homologous domain family bunch for a plurality of pieces of homologous domain family information belonging to a plurality of the classification categories on the basis of homologous domain family information regarding the families of homologous string pieces and content of the homologous string pieces belonging to the families included in a result of phylogeny analysis by utilizing the result of phylogeny analysis performed on the basis of positions where the homologous string pieces exist and homological relationship of the homologous string pieces while attention is focused on the homologous string pieces within a string for string data representing the string including one or more characters.

7. A program for causing a computer to realize
a homologous domain family bunch classifying function of classifying a group of families having homology across classification categories as a homologous domain family bunch for a plurality of pieces of homologous domain family information belonging to a plurality of the classification categories on the basis of homologous domain family information regarding the families of homologous string pieces and content of the homologous string pieces belonging to the families included in a result of phylogeny analysis by utilizing the result of phylogeny analysis performed on the basis of positions where the homologous string pieces exist and homological relationship of the homologous string pieces while attention is focused on the homologous string pieces within a string for string data representing the string including one or more characters.
